(19) United States
(12) Patent Application Publication
(10) Pub. No.: US 2021/0335073 A1
(43) Pub. Date: Oct. 28, 2021

(54) Identification and Contact Tracing System and Method

(71) Applicant: Paul K Luker LLC, Carthage, TX (US)

(72) Inventor: Paul Kimbel Luker, Carthage, TX (US)

(73) Assignee: Paul K Luker LLC, Carthage, TX (US)

(21) Appl. No.: 17/301,573

(22) Filed: Apr. 8, 2021

Related U.S. Application Data
(60) Provisional application No. 63/014,198, filed on Apr. 23, 2020.

(51) Int. Cl. G07C 9/25; G07C 9/29; G06K 9/00; G16H 10/60; H04N 5/33

(52) U.S. Cl. CPC G07C 9/257; G07C 9/29; H04N 5/33; G06K 9/00281; G16H 10/60; G06K 9/00288

(57) ABSTRACT

Described herein is a system and method for creating a chain of trust to determine whether to grant an individual access to a particular business, area, venue or site. The system and method use an identification card that is issued to an individual. The identification card may contain a near field communication chip that can be read by a smart phone or tablet. The smart phone or tablet also communicates with a temperature scanner to record the individual's body temperature. The smart phone or tablet may also contain a facial recognition module to verify an individual's identity.

### BACKGROUND

### 1. Field of the Invention

The present disclosure is generally directed to a method and a system that measures and identifies characteristics of an individual to determine whether the individual should be granted access to a particular place, business, venue or area. The disclosed method and system can also broadly be used to identify individuals with certain characteristics of interest, communicate such information to appropriate agencies or other entities and provide for contact tracing of such individual. More particularly, the present disclosure is consistent with providing an integrated, comprehensive method and system for identifying and reporting certain physical characteristics of an individual in order to determine whether that person should be granted access to a particular place, business, venue or area and/or to determine whether contract tracing of the individual should be performed.

### 2. General Background of the Invention

Various systems have been proposed to provide for attempting to provide secure access to facilities. U.S. Patent Publication No. 2018/0047237, entitled WORKSITE INGRESS/EGRESS SYSTEM and assigned to the present applicant, describes a system that uses various criteria to determine whether to allow personnel access to a secured worksite. U.S. Patent Publication No. 2018/0047237, entitled WORKSITE INGRESS/EGRESS SYSTEM is incorporated by reference in its entirety herein. While this system and method is useful in certain situations, it does not provide a way to obtain physical characteristics, e.g., an individual's temperature, and use that information to determine whether to allow the person to access a facility.

Other systems, such as U.S. Pat. No. 6,049,776 invented by Donnelly and U.S. Pat. No. 8,660,876 invented by Woodings et al. disclose various controlled access systems, but these systems fail to provide sufficient real time information, and only use a snap shot of information, which is often outdated, to determine whether to allow access.

### SUMMARY OF THE INVENTION

The inventions disclosed herein describe a system and method for creating a chain of trust to determine whether to grant an individual access to a particular business, area, venue or site. The system and method use an identification card that is issued to an individual. The identification card may contain a near field communication chip that can be read by a smart phone or tablet. The smart phone or tablet also communicates with a temperature scanner to record the individual's body temperature. The smart phone or tablet may also contain a facial recognition module, a facial recognition scanner (reader) or device, a finger print recognition module, a finger print scanner (reader) or device, an iris scan module, an iris scanning device (reader) or other biometric verification technology to verify an individual's identity. These biometric readers and modules may also be separate from the smart phone or tablet. In operation, an individual wishing to gain access to a site presents an identification card. The identification card is read by the smart phone or tablet, and information regarding the individual is acquired. A temperature scanner in communication with the smart phone or tablet reads the individual's temperature and records it. If the individual's temperature is within specified parameters, the individual may be granted access to the site. Facial recognition or other biometric information may also be used to confirm the individual's identity, and a determination of whether to grant access may be made based on whether the facial recognition or other biometric information passes or fails. An additional aspect may include a system that can verify whether the individual presenting the identification card has been vaccinated for a particular predetermined illness or group of illnesses. Access to the site may also be based, in whole or in part, based on the vaccination check performed by the system.

### BRIEF DESCRIPTION OF THE DRAWINGS

FIG. 1 is a system topology in accordance with the present disclosure.

### DETAILED DESCRIPTION

The present disclosure relates to a system and method that can be used to identify certain physical characteristics of individuals in order to determine whether to allow the particular individual to have access to a particular business, worksite, venue or area. Further, the system and method allows for the reporting of the physical characteristic of an individual to an appropriate agency to inform the agency so that the agency can conduct contract tracing. Important advantages of the invention are that enhances the ability to do contract tracing, which can be used to mitigate the community spread of diseases, such as Covid-19. The system provides for the secure identification of a person in combination with a remote temperature scanner that can scan a person to determine their temperature. The remote scanning allows for appropriate distancing to prevent or mitigate potential community disease spread. Upon collecting a person's temperature, the system and method provides for a secure and immediate report to appropriate health department officials, so that the health officials can determine whether to conduct individual contact tracing.

The system operates in a cloud-based computing environment and allows for access to multiple databases, at the same or different locations, to determine whether to allow access by an individual, and to report information to appropriate organizations.

The system and method optionally uses a card that with a near field communication chip that is presented to an electronic device, such as a smart phone or tablet, that is equipped with a near field chip reader to access information about an individual from the card. The smart phone or tablet may contain or be equipped to communicate with a mechanism to read an individual's temperature, such as an infrared temperature scanner. Other known types of scanners or devices to read an individual's temperature may also be used. The smart phone or tablet may also contain a facial recognition module, a facial recognition scanner (reader) or device, a finger print recognition module, a finger print scanner (reader) or device, an iris scan module, an iris scanning device (reader) or other biometric verification technology to verify the identity of the individual presenting the identification card. These biometric readers and modules may also be separate from the smart phone or tablet.

For the purposes of promoting an understanding of the principles of the invention, reference will now be made to certain embodiments thereof and specific language will be used to describe the same. It will nevertheless be understood that no limitation of the scope of the invention is thereby intended, such alterations, further modifications and further applications of the principles of the invention as described herein being contemplated as would normally occur to one skilled in the art to which the invention relates.

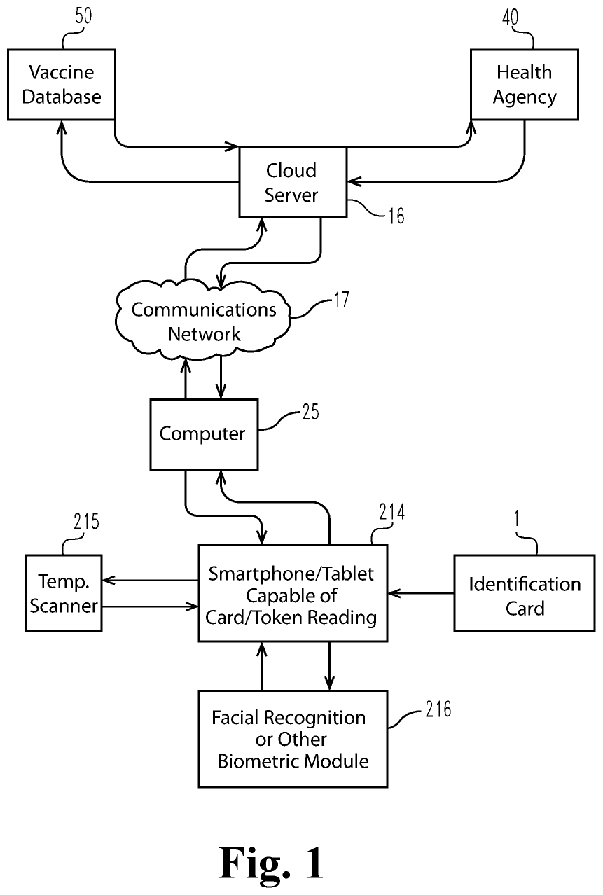

The preferred system is shown in FIG. 1. An individual presents an identification card (1) to a smart phone or tablet (214) that is capable of reading a near field communication chip in the identification card. The smart phone or tablet (214) is also capable of reading a token presented with the identification card. A temperature scanner (215) is in communication with the smart phone or tablet (214) and can be used to scan an individual to determine their body temperature. The smart phone or tablet is also in communication with a facial recognition module (216) that can be used to verify an individual's identity. The smart phone or tablet (214) is in communication via a communications network with a cloud server (16), a vaccine database (50) and one or more health agencies (40).

Individuals are issued a digital identification card (1) that is accessed by a smart phone or tablet containing an application. The digital identification card (1) used in the system and method are described in U.S. Patent Publication No. 2018/0047237, entitled WORKSITE INGRESS/EGRESS SYSTEM, which is incorporated by reference in its entirety herein.

Enrollment of business, worksites, venues, or other areas or entities can be performed online. When the individual's identification card (1) is scanned it creates a point of verification that is recorded in the system. This point of verification is a snapshot of the card activity that records the individual's temperature at that specific instance.

An individual's body temperature is a leading indicator of a possible infection. Upon presentment of an individual's identification card (1) to the smart phone or tablet (214), in combination with an infrared thermometer (215) can record the individual's temperature. These infrared thermometer devices (215) can be accessed without exposing any site personnel to a possible contagion because the temperature monitor is remote to the personnel. This ensures the safety of the site personnel while achieving the goal of measuring the body temperature of the person seeking access to the site. The temperature monitors are calibrated to the desired temperature, below which access would be granted. If the measured temperature of the person seeking entrance exceeds the calibrated setting the device will alarm. The person triggering the alarm will then be directed to additional medical evaluation and denied access to the site. If the measured temperature of the person seeking entrance is within the calibrated setting, the device may verify the individual's identity using the facial recognition module (216). This facial recognition step could also be performed prior to any temperature scan of the individual. If the facial recognition step fails at any point, the individual may be denied access to the site.

The combination of the identification card system and the temperature measurement process can quickly assess the person requesting entry to the site. Any exception to the protocol can produce an immediate notification via the system to notify health department officials at one or more health agencies (40) of the identity and measured temperature of the person requesting site access. This initiates the contact tracing and an immediate response can be initiated to contact this person for further evaluation.

The identification card (1) can also link to a database (50) containing data regarding whether the individual presenting the identification card has been vaccinated for a particular illness, such as Covid-19. This information can also be used in evaluating whether the individual is granted access to the site. The system also has reporting features that can query and filter results for creating a history of activity based on the person's card activity which presents the opportunity to look back at the history of the person manifesting symptoms to identify possible exposures before they became symptomatic. The history of activity can be stored on site at a local computer (25) or in a cloud server (16). Contact tracing will be much more effective if real data can be evaluated.

While the invention has been illustrated and described in detail in the drawings and foregoing description, the same is to be considered as illustrative and not restrictive in character, it being understood that only the preferred embodiments have been shown and described and that all changes and modifications that come within the spirit of the invention are desired to be protected.

What is claimed is:

1. An apparatus for controlling access to an area comprising:
an access card containing information identifying an individual including biometric information;
an access card reader;
a temperature scanner capable of measuring the temperature of an individual, the temperature scanner being in communication with a computer;
a biometric reader capable of scanning a biometric feature of an individual;
a computer capable of determining whether the scan from the biometric reader matches an individuals biometric information as read by the access card reader;
a computer with a stored preset temperature limit that determines whether to allow an individual access to an area based on a comparison of the preset temperature limit and the temperature as read by the temperature scanner.

2. The apparatus for controlling access to an area of claim 1 wherein the temperature scanner is an infrared temperature scanner.

3. The apparatus for controlling access to an area of claim 1 wherein at least one of the computers is a smart phone.

4. The apparatus for controlling access to an area of claim 1 wherein at least one of the computers is a tablet computer.

5. The apparatus for controlling access to an area of claim 1 wherein the biometric reader is a facial recognition reader.

6. The apparatus for controlling access to an area of claim 1 wherein the biometric reader is an iris recognition reader.

7. The apparatus for controlling access to an area of claim 1 wherein the biometric reader is a finger print reader.

8. An apparatus for controlling access to an area comprising:
an access card containing information identifying an individual including biometric information;
an access card reader;
a biometric reader capable of scanning a biometric feature of an individual;
a computer capable of determining whether the scan from the biometric reader device matches the individuals biometric information as read by the access card reader;
a computer capable of querying a database containing information about an individuals vaccination records;
a computer that determines whether to allow an individual access to an area based on an inquiry of the database containing individual vaccination records and based on a comparison of the biometric information of the individual and biometric information as read by the access card reader.

9. The apparatus for controlling access to an area of claim 8 wherein at least one of the computers is a smart phone.

10. The apparatus for controlling access to an area of claim 8 wherein at least one of the computers is a tablet computer.

11. The apparatus for controlling access to an area of claim 8 wherein the biometric reader is a facial recognition reader.

12. The apparatus for controlling access to an area of claim 8 wherein the biometric reader is an iris recognition reader.

13. The apparatus for controlling access to an area of claim 8 wherein the biometric reader is a finger print reader

14. A method for controlling access to an area comprising:
reading an access card with a card reader containing biometric information of an individual;
scanning and recording a body temperature of the individual presenting the access card;
scanning the individuals biometric information;
communicating the individuals body temperature and biometric information with a computer;
verifying the individuals biometric information match the individuals biometric information as read from the access card;
comparing the individuals body temperature with a preset body temperature limit stored in a computer to determine whether to grant the individual access to an area.

15. The method according to claim 14 wherein the computer is a smart phone.

16. The method according to claim 14 wherein the computer is a tablet computer.

17. A method for controlling access to an area comprising:
reading an access card with a card reader containing facial recognition information of an individual;
scanning the individuals facial features;
verifying the individuals scanned facial features match the individuals facial features as read from the access card;
using a computer to query a database containing information about the individuals vaccination records;
using a computer to determine whether to allow an individual access to an area based on an inquiry of the database containing individual vaccination records and based on the verification of the individuals facial recognition information as read by the access card reader.
